(19) United States
(12) Patent Application Publication
(10) Pub. No.: US 2015/0367064 A1
(43) Pub. Date: Dec. 24, 2015

(54) Platelet Concentrating System

(71) Applicant: Patrick Pennie, Fort Myers, FL (US)

(72) Inventor: Patrick Pennie, Fort Myers, FL (US)

(21) Appl. No.: 14/743,692

(22) Filed: Jun. 18, 2015

Related U.S. Application Data
(60) Provisional application No. 62/013,870, filed on Jun. 18, 2014.

(51) Int. Cl. A61M 1/36

(52) U.S. Cl. CPC A61M 1/3696

(57) ABSTRACT

A method for more effectively concentrating blood platelets for use in medical procedures includes providing preparation and concentrating tubes. Anticoagulated whole blood is added to the preparation tube, which is centrifuged to separate red blood cells from a platelet plasma suspension The platelet plasma suspension is aspirated and loaded into the concentrating tube, which is itself centrifuged to separate the platelet plasma suspension into platelet poor plasma and platelet rich plasma layers. The platelet poor plasma is aspirated through a port of the concentrating tube and the remaining concentrated PRP is aspirated through the same or a different port of the concentrating tube.

### FIELD OF THE INVENTION

This invention relates to a platelet concentrating system and, more particularly, to a system and method for producing platelet rich plasma (PRP) to be used in medical applications. The system and method increase the concentration of platelets and reduce the level of red blood cells in the PRP.

### BACKGROUND OF THE INVENTION

Platelet-rich blood plasma, commonly known as PRP, is widely used in a variety of medical procedures. The worldwide demand for this blood product is ever increasing. PRP exhibits particularly effective growth-promoting features, which are particularly beneficial in medical applications such as wound care, bone regeneration, maxiofacial surgery and dental care. Platelet rich plasma is most effective when it utilizes a high concentration of blood platelets.

Various conventional devices and processes are available for separating a whole blood sample into its constituent parts (i.e. plasma, red blood cells and platelets). Conventionally, the blood sample is centrifuged and the platelets, combined with white blood cells in the form of a whitish buffy coat, are separated from the blood sample and sequestered in concentrated form though aspiration. Traditional, aspiration techniques often failed to provide a satisfactory concentration of platelets for achieving optimal medical benefits. In addition, cross contamination between the constituent blood components was frequently encountered.

Recently, I have jointly invented and developed products which have significantly improved platelet concentration while reducing cross contamination of blood constituents. See U.S. Pat. Nos. 6,835,353 and 7,976,796. These products have facilitated and improved the process of manufacturing high quality, concentrated platelet rich plasma. Nonetheless, excessive levels of red blood cells commonly remain in much of the PRP currently produced. To date, the PRP industry has experienced difficulty obtaining red blood cell concentrations of less than 15% using conventional processing techniques. It would be both medically and economically desirable to reduce red blood cells while increasing platelet concentrations and purity in processed PRP. The platelet rich buffy coat produced by conventional PRP processing techniques typically contains a significant amount of contaminating red blood cells, even after the blood sample has been separated into its constituent parts.

A further problem accompanying the standard manner of producing PRP involves the anticoagulant that is used. An anticoagulant in the form of ACDA is typically added to the processed blood product in order to restrict clotting and allow the product to be effectively separated into its constituent components. Although ACDA is a fairly effective anticoagulant, it is quite acidic and tends to cause painful side effects for the patient being treated by the manufactured PRP. A great need exists for the use of a different anticoagulant in the production of PRP, which will work at least as efficiently as ACDA, but with less painful side effects.

### SUMMARY OF THE INVENTION

It is therefore an object of the present invention to provide a platelet concentrating system and related method that enable platelet concentrated PRP to be manufactured more effectively and efficiently than has heretofore been achieved using known technology.

It is a further object of this invention to provide a system and technique for removing more red blood cells from platelet rich plasma so that a more highly concentrated and medically effective PRP product is achieved.

It is a further object of this invention to provide a platelet concentrating system that utilizes commercially available PRP processing equipment in a unique and efficient manner and does not require the development or purchase of complicated, expensive untested and/or experimental technology.

It is a further object of this invention to provide a platelet concentrating system and related method which enable the manufacture of improved, highly concentrated PRP in a relatively uncomplicated, quick, efficient, safe and effective manner.

It is a further object of this invention to provide a platelet concentrating system and related method which employ an anticoagulant that is at least as effective and far less painful than conventional anticoagulants conventionally used to produce platelet-rich plasma.

This invention results from a realization that a high quality, medically effective platelet rich plasma with an increased concentration of platelets may be obtained by processing a patient's blood in multiple stages using the centrifuge tube disclosed in U.S. Pat. No. 7,976,796 (hereinafter Patent No. '796) and a second centrifuge tube featuring two aspiration ports, either as disclosed herein or as disclosed in U.S. Pat. No. 8,835,353 (hereinafter Patent No. '353). The process also employs conventional items such as aspirating syringes and a centrifuge machine. This invention also results from a realization that sodium citrate is especially effective to use as an anticoagulant in the production of PRP. Sodium citrate is less acidic than ACDA, which is conventionally used in PRP manufacture, and therefore causes less painful side effects in patients treated with platelet rich plasma. To date, ACDA has been universally utilized in PRP production because of ACDA's high dextrose content, which allows the PRP to be stored for extended periods. However, PRP is usually manufactured and used at the point of care and thus does not require extended storage. Sodium citrate, which lacks the dextrose content of ACDA, can therefore be used as an effective and far less painful substitute.

This invention features a platelet concentration system and a related method for producing platelet rich plasma (PRP) having a reduced level of red blood cells and an increased platelet concentration. A first preparation centrifuge tube and a second concentrating centrifuge tube are provided. The preparation tube includes an elongate receptacle having an interior chamber for receiving a blood product therein. The receptacle has closed upper and lower ends and a side wall extending between the upper and lower ends. A common inlet and outlet port is formed in the upper end of the receptacle and a flexible, fluid conducting pipe is communicably connected to the common port for extending through the chamber. A liquid-impermeable sealing diaphragm is mounted for sliding longitudinally through the chamber of the receptacle and maintaining sealing interengagement with an interior surface of the side wall of the receptacle. The flexible pipe is disposed through the diaphragm in communication with the region of the chamber located below the diaphragm. The concentrating tube includes a receptacle having an interior chamber, which receptacle has closed upper and lower ends and a side wall extending between the upper and lower ends. Plasma and PRP aspiration ports are formed in the receptacle proximate the upper end of the receptacle. The plasma aspiration port has an aspiration pipe attached communicably thereto and extending through the interior chamber of the receptacle.

To perform the process of this invention, a whole blood sample is drawn from a patient and mixed with an anticoagulant to provide an anticoagulated blood mixture. The anticoagulated blood mixture is introduced into the preparation tube through the common port and flexible pipe such that the mixture enters the chamber of the preparation tube below the diaphragm to drive the diaphragm upwardly within the chamber. The preparation tube is then centrifuged at a speed and duration that separates that anticoagulated whole blood sample into discrete upper and lower fluid layers in the chamber of the preparation tube receptacle. The upper layer includes primarily a platelet plasma suspension that retains at least 30% of the platelets from the blood sample. The lower layer includes primarily red blood cells. The upper fluid layer is aspirated from the interior chamber of the preparation tube receptacle through the flexible pipe and the common port and is then introduced into the interior chamber in the receptacle of the concentrating tube through the plasma port of the concentrating tube. The concentrating tube is then centrifuged at a speed and duration that separates the platelet plasma suspension into discrete top and bottom layers in the chamber of the concentrating tube receptacle. The top layer includes a platelet poor plasma retaining less than 50% of the platelets from the platelet plasma suspension and the bottom layer includes a platelet rich plasma in the form of a whitish buffy coat retaining more than 50% of the platelets from the platelet plasma suspension. The second centrifuging step strips additional red blood cells from the platelet plasma suspension and deposits those red blood cells into the top layer platelet poor plasma. The aspiration pipe attached to and extending downwardly from the plasma port of the concentrating tube is positioned such that its lower end is located within the top fluid layer and above the platelet concentrated buffy coat layer. The platelet poor plasma is next aspirated from the concentrating tube receptacle through the aspirating pipe and attached second port. The concentrating tube is then gently agitated, such as by swirling or otherwise, to resuspend the platelets remaining in the bottom fluid layer into the remaining plasma. Plasma may be added to the concentrating tube through the plasma port to obtain additional volume if required. Finally, the concentrating tube is inverted and the highly concentrated PRP that remains in the concentrating tube receptacle is aspirated through the second, PRP port of the concentrating tube. The “pure” PRP that is retrieved in this manner will typically exhibit a trace red blood cell concentration of less than one percent, which is far less than is achieved using conventional PRP production techniques.

In a preferred embodiment, the anticoagulant may include sodium citrate, which is typically mixed with a patient's blood in a ratio of 1:5. Preferably, the whole blood sample and the anticoagulant are mixed in a 60 ml syringe and the anticoagulated mixture is loaded into the preparation tube for the initial centrifuging step. The anticoagulated whole blood may be centrifuged for about 1.5 minutes at a speed of approximately 3800 RPM.

The common port of the preparation tube and the plasma and PRP ports of the concentrating tube may include releasable closures or caps, which are attached to the ports during the centrifuging steps and which are removed for loading or aspiration of the fluids into and out of the tubes.

After the initial centrifuge step, the cap attached to the closed port of the preparation tube receptacle is removed and the upper fluid layer containing the plasma platelet suspension is aspirated into a 60 ml plasma syringe. When a 60 ml anticoagulated whole blood sample is involved, the aspirated suspension typically has a volume of approximately 35 ml. The platelet plasma suspension is transferred to the concentrating tube receptacle through the open plasma port of the concentrating tube. Typically, both the plasma and PRP ports of the concentrating tube are formed through the upper end of the concentrating tube receptacle. After the platelet plasma suspension (which includes some remaining red blood cells) is loaded into the chamber of the concentrating tube receptacle, both the plasma and PRP ports are capped. The concentrating tube may then be centrifuged at 3800 RMPs for a duration of 5 minutes. Typically during each centrifuge step the tube that is being centrifuged is counterbalanced.

After the second centrifuge step is completed, the plasma port of the concentrating tube is opened and the top, platelet poor plasma layer is aspirated from the chamber of the centrifuge tube receptacle through the aspiration pipe and plasma port. Approximately, 7 ml of platelet concentrated buffy coat PRP remain in the bottom fluid layer of the concentrating tube, along with trace levels of red blood cells. At this point, a 12 ml syringe may be attached to the PRP port and the concentrating tube is gently swirled to resuspend the concentrated platelets in the remaining plasma. Finally, the concentrating tube is inverted and the highly concentrated PRP remaining in the tube is aspirated through the PRP port. The 12 ml syringe is then removed and capped. The concentrated or purified PRP is then ready for use in a variety of medical applications.

An alternative concentrating tube in accordance with this invention may include a single, common inlet and aspiration port formed through an upper end portion of the concentrating tube receptacle. An aspiration pipe may be communicably connected to the port and extend downwardly through a chamber of the concentrating tube receptacle. A lower end of the aspiration pipe may carry a distal nozzle. After the platelet plasma suspension is aspirated from the preparation tube, it is introduced into the chamber of the concentrating tube through the common port. The lower end of the aspiration pipe and distal nozzle are positioned within the chamber such that after the concentrating tube is centrifuged, the nozzle is proximate the bottom of the platelet poor plasma layer. Following centrifugation of the concentrating tube, the platelet plasma suspension is separated into an upper platelet poor plasma layer and a lower platelet rich plasma layer as in the previous version. The platelet poor plasma is aspirated through the nozzle, aspiration tube and single port. The remaining platelet rich plasma is swirled or otherwise agitated and additional plasma is added if desired. The concentrating tube is then angularly tilted to immerse the nozzle in the platelet rich plasma and the remaining platelet rich plasma is aspirated through the nozzle. Preferably, the aspiration pipe is flexible and the nozzle is angled toward the side wall of the receptacle so that aspiration of the remaining PRP is facilitated.

### BRIEF DESCRIPTION OF THE DRAWINGS

Other objects, features and advantages will occur from the following description of a preferred embodiment and the accompanying drawings, in which:

FIG. 1 is a block diagram depicting a preferred method for concentrating platelets according to this invention;

FIG. 2 is a front elevational view of a preferred preparation tube used in the platelet concentration system of this invention;

FIG. 3 is a front elevational view of a preferred concentrating tube used in this invention;

FIG. 4 is a front elevational view of a preferred anticoagulant being drawn into a 60 ml loading syringe for use in the platelet concentrating process;

FIG. 5 is a front elevational view of the loading syringe, which has drawn a patient's whole blood sample and mixed the blood sample with the anticoagulant;

FIG. 6 is a front elevational view of the anticoagulated whole blood being loaded into the preparation tube.

FIG. 7 is a front elevational view depicting the discrete platelet plasma suspension (PPS) and red blood cell (RBC) layers in the preparation tube following centrifugation of the preparation tube; a 60 ml syringe is operatively engaged with the common port of the preparation tube to aspirate the platelet plasma suspension from the tube;

FIG. 8 is a front elevational view of the syringe engaged with the plasma port of the concentrating tube for transferring the platelet plasma suspension into the chamber of the concentrating tube;

FIG. 9 is a front elevational view of the concentrating tube after a second centrifuge step has been performed and wherein the platelet plasma suspension has been separated into an upper layer of platelet poor plasma and a lower layer of platelet concentrated buffy coat/platelet rich plasma;

FIG. 10 is a front elevational view of a 60 ml syringe engaged with the plasma port of the concentrating tube for aspirating platelet poor plasma (PPP) from the tube;

FIG. 11 is a front elevational view of the concentrating tube in an inverted condition and with a 12 ml. syringe engaged with the PRP port for aspirating the concentrated PRP product from the concentrating tube at the completion of the process;

FIG. 12 is a front elevational view of an alternative preparation tube that can be used in this invention;

FIG. 13 is a front elevational view of an alternative concentrating tube that can be used in this invention; and

FIG. 14-17 are front elevational views of the PPP and PRP layers being sequentially aspirated from the concentrating tube of FIG. 13 in accordance with the process of this invention.

### DETAILED DESCRIPTION OF PREFERRED EMBODIMENTS

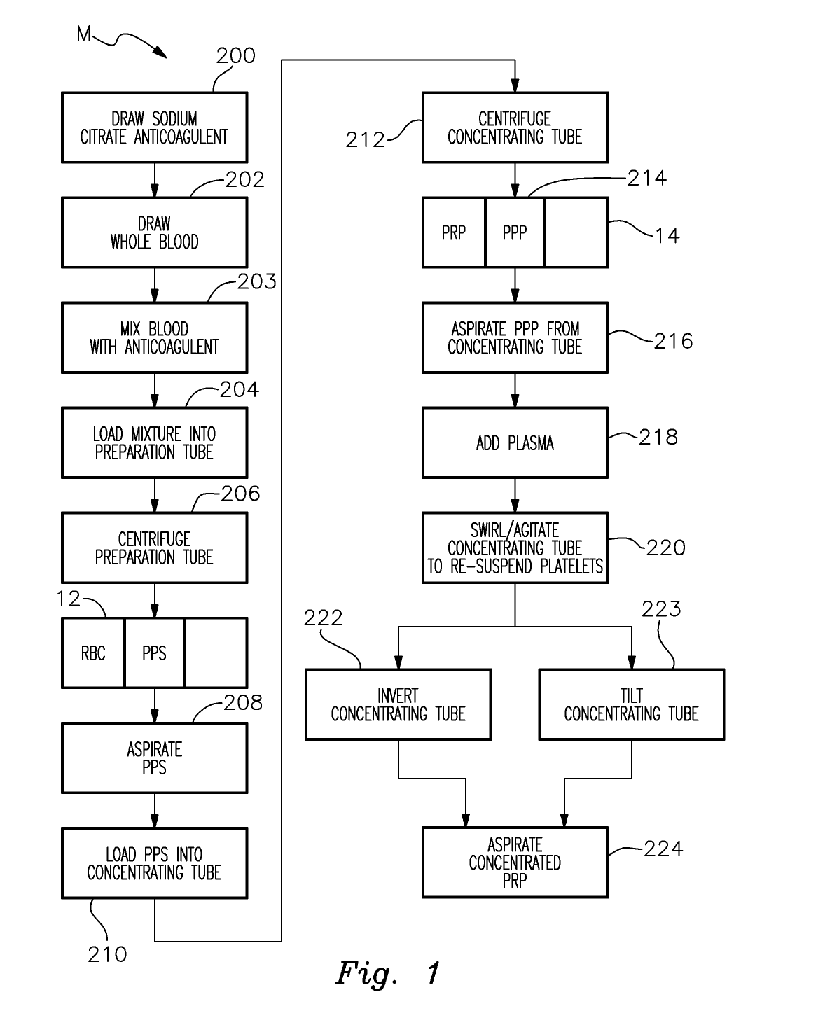

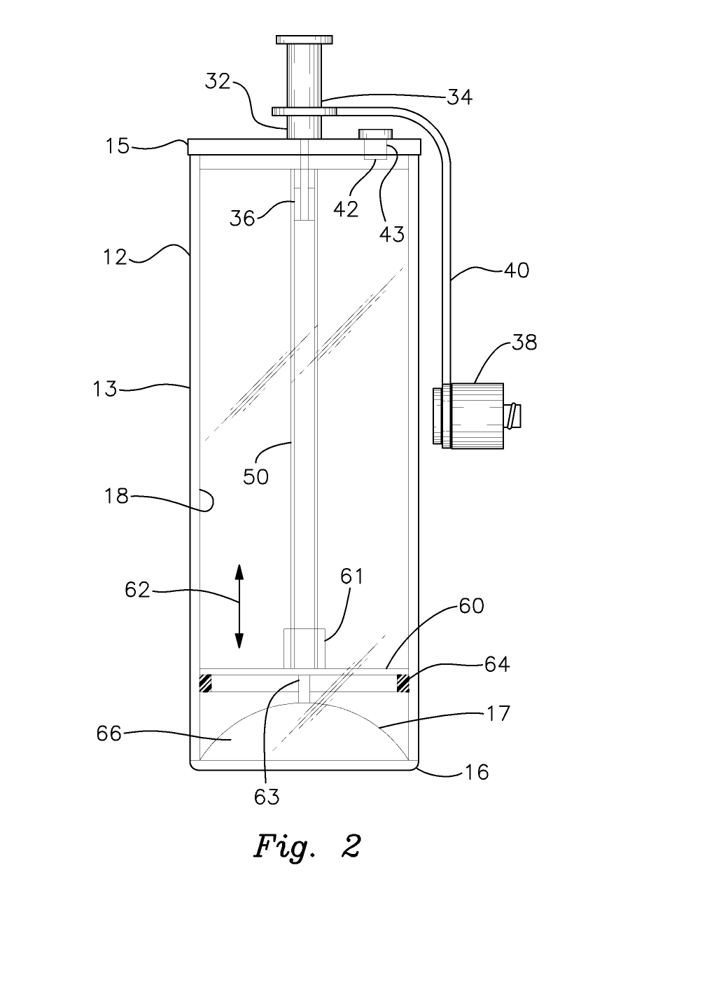

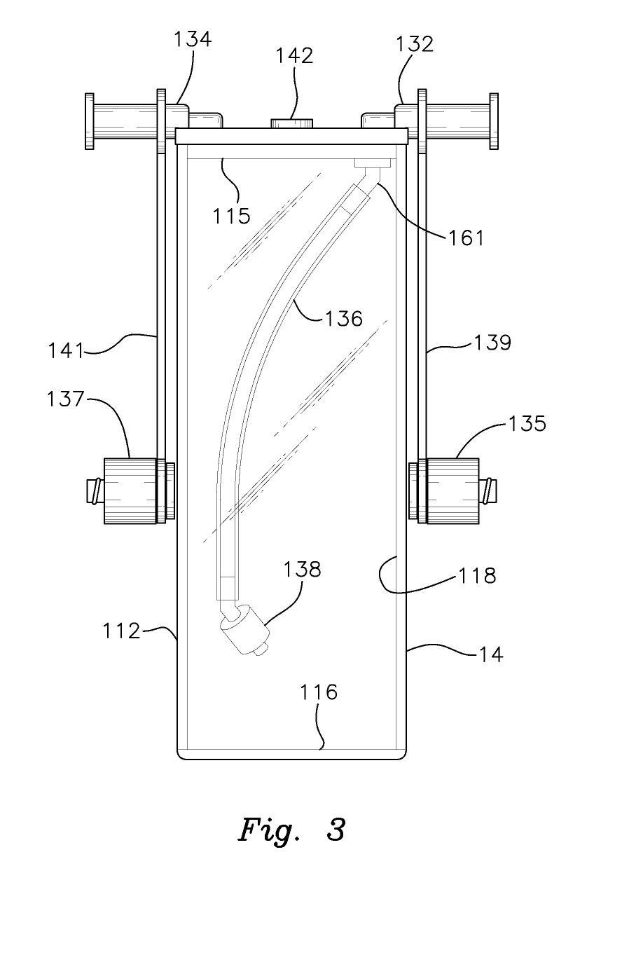

There is shown in FIG. 1 a block diagram illustrating a method of concentrating blood platelets to provide a more concentrated and effective platelet-rich plasma for use in wound healing, oral surgery, bone regeneration and various other medical procedures. The method is performed using a novel combination of various conventional devices, which are currently available and previously used in the manufacture of PRP as well as newly modified and improved devices for use in practicing this method. These devices more specifically include a preparation tube 12, shown alone in FIG. 2, and a concentrating tube 14, shown alone in FIG. 3, as well as other standard components including syringes and centrifuges, which are previously employed in this field. The construction and operation of preparation tube 12 are largely disclosed in US Patent No. '796. The Genesis™ Centrifuge Tube manufactured by Emcyte Corporation is especially effective preparation tube for use in the present invention. An alternative preparation tube may include the Progenikine™ centrifuge tube, which is the subject of U.S. patent application Ser. No. 14/741,920. At least some of the operating principles of concentrating tube 14 are described in US Patent No. '353, although the particular construction of tube 14, as disclosed herein, is newly developed and that tube is used in a novel and unique manner in accordance with the details and principles of this invention. In alternative embodiments, for example, the Sequire™ Centrifuge Tube manufactured by Emcyte Corporation may be utilized as the concentrating tube. The specifications and descriptions contained in Patent Nos. '796 and '353 and Ser. No. 14/741,920 are incorporated herein by reference. It should be understood that the preparation and concentrating tubes described herein may alternatively employ structure and function as disclosed in the referenced patents. The tubes may also feature modifications to and variations of the disclosed centrifuge tube structures, which are within the scope of the referenced patents and/or are obvious to persons skilled in the art.

Tubes 12 and 14 comprise centrifuge tubes. As used herein, “centrifuge tube” or “tube” should be understood to comprise various shapes and sizes of vessels, receptacles and containers having an interior chamber for holding a fluid biological product, such as blood, and capable of being centrifuged to separate the product into constituent components. When whole blood is involved, the constituent components are usually red blood cells, plasma and platelets, which are typically mixed with white blood cells in the form of a whitish “buffy” coat. The centrifuge tubes are not limited to just tubular and elongate configurations, although such configurations will typically be used in preferred embodiments of this invention.

As depicted in FIG. 2, preparation tube 12 includes a tubular or cylindrical receptacle 13 having a permanently capped or closed upper end 15. A flat base 16 is similarly formed at the lower end of tubular receptacle 13 for supporting the tubular receptacle in an upright condition on a table or other flat or horizontal surface. In this way, the preparation tube does not require a separate rack or holder for support.

Tubular receptacle 13, as well as permanently capped upper end 15 and base 16 are typically composed of a durable plastic material such as polypropylene or other material suitable for medical or veterinary applications. Each of the tubes disclosed herein may comprise similar materials. The tube should likewise be constructed to withstand the force exerted by centrifuging. In certain applications, shatter resistant glass may be employed. Although the tube is preferably formed with a permanently capped upper end, in alternative embodiments, a removable (e.g. threadabe) cap may be utilized. Various alternative and/or analogous forms of construction are disclosed in US Patent No. '353.

Tubular receptacle 13 includes an interior chamber 18 that extends from upper end 15 to base 16. Chamber 18 accommodates blood (or, in other applications, chemicals, stem cells, bone marrow aspirant and other biological fluids/products) to be centrifuged and aspirated using tube 12.

In the version disclosed herein, receptacle 13 does not include graduated markings along the exterior side wall of the receptacle. In alternative embodiments, various types of graduated markings representing fluid volume may be formed along this side wall.

A common inlet/outlet port 32 is formed unitarily in upper end 14 of tubular receptacle 13. Port 32 includes a central opening that extends through upper end 14. An upper end of port 32 or stem 34 is disposed exteriorly of the tubular receptacle, and a lower end 36 of the port is disclosed interiorly of chamber 18. Lower end 36 may comprise a fitting or receptacle for operatively receiving a syringe that is used to introduce a whole blood product into chamber 18 of tube 12. Preferably, common inlet/outlet port 32 is composed of material similar to that forming the tube itself. The common port may be molded together with the tube in a single manufacturing process. Various alternative types of inlet/outlet ports may be employed including Leur™-type ports as are described in referenced Patent Nos. '353 and '796. A removable closure 38 is secured to the outer stem 34 of port 32 by a connecting strap 40. During the centrifuging operation, as well as at other times when fluid is not being introduced into or removed from tube 12, closure 38 is engaged with the upper stem 34 of port 32 to maintain the port in a closed condition. This is represented by the engaged closure 38a in FIG. 2.

A vent 42 is formed through upper end 15 adjacent common port 32. The vent maintains a stable neutral pressure within tubular receptacle 13 during the aspiration process. Vent 42, which is shown in FIG. 2 as being closed by a removable cap 43, may be formed at various locations at the capped upper end of the tube.

An elongate, flexible pipe 50 is communicably engaged with the interior fitting 36 of port 32. The pipe is composed of a flexible, yet strong plastic material. Silicone or other flexible plastic material is especially suited for the pipe. Prior to the use of tube 12, pipe 50 features the elongate and relatively straight condition illustrated in FIG. 2. As is described more fully below, when a whole blood sample is loaded into tube 12, pipe 50 is caused to flex or collapse by a disk-shaped piston or diaphragm 60 attached to the lower or distal end of pipe 50. Diaphragm 60 is itself slidably mounted for longitudinal movement within chamber 18 of receptacle 13. As described in Patent No. '796, diaphragm 60 preferably has a circular, disk-like shape with a peripheral edge that sealingly and slidably interengages the interior wall of tubular receptacle 13. The diaphragm includes a peripheral O-ring seal 64 that provides the sealing interengagement. In the version disclosed herein, the diaphragm has a concave bottom surface 17. Diaphragm 60 is movable longitudinally within chamber 18 as indicated by double headed arrow 62 in FIG. 2. Diaphragm 60 has a cylindrical fitting 61 that is attached to and extends upwardly from an upper surface of the diaphragm. Fitting 61 receives a lower end of pipe 50. A tubular channel 63 is communicably connected to a lower end of pipe 50 and itself extends transversely through diaphragm 60. The lower end of channel 63 communicates with an interior portion 66 of chamber 18 located below diaphragm 60.

In other versions, the preparation tube may eliminate an aspiration pipe altogether. See Ser. No. 14/741,920. In such cases a vent is typically formed in a bottom portion of the tube for neutralizing pressure within the tube. The precise positioning of the common inlet and outlet port and the vent may be varied within the scope of this invention. It is critical that the common inlet and outlet port and the vent communicate with different regions of the chamber separated by the diaphragm.

As shown in FIG. 3, concentrating tube 14 includes a tubular or cylindrical receptacle 112 with permanently capped upper and lower ends 115 and 116 respectively. Lower end 116 is again substantially flat so that the tube can stand upright on a table or other planar supportive surface. Tube 14 may be composed of materials similar to those used to manufacture preparation tube 12 and as further described in Patent Nos. '353 and '796. It should also be understood that the permanently closed upper end 115 may again be substituted with a removable closure or cap. In either case, receptacle 112 includes an interior chamber 118 that extends between upper end 115 and lower end 116. Once again, the chamber accommodates a fluid (i.e. blood product constituents) being centrifuged. Graduated markings, not shown in FIG. 3, may optionally be employed on the cylindrical side wall of tube 14.

A first plasma aspiration port 132 and a second PRP aspiration port 134 are formed through upper end 115. The ports may comprise Leur™ type ports that are formed unitarily through upper end 115 in the manner shown in FIG. 3. Alternatively, separate and distinct port components may be fitted through and secured within respective openings in upper end 115. The exterior ends of ports 132 and 134 extend radially or transversely in opposite directions from the upper end 115 of tube 112. The ports may comprise Leur™ type fittings. Respective removable closures 135 and 137 are attached to the exterior portions of ports 132 and 134 by connecting straps 139 and 141. Caps 135 and 137 may be engaged with respective ports 132 and 134 to close those ports, as indicated by attached caps 135x and 137x. In alternative versions, one or both of ports 132 and 134 may be formed elsewhere in an upper end portion of the receptacle, i.e. through the side wall 118 proximate upper end 115.

A vent cap 142 is removably received in a 1-2 mm vent hole formed through upper end 115. This vent hole is selectively opened to maintain a stable neutral pressure within receptacle 112 during the aspiration process described below. The vent may be formed at various locations in the upper end of the tube.

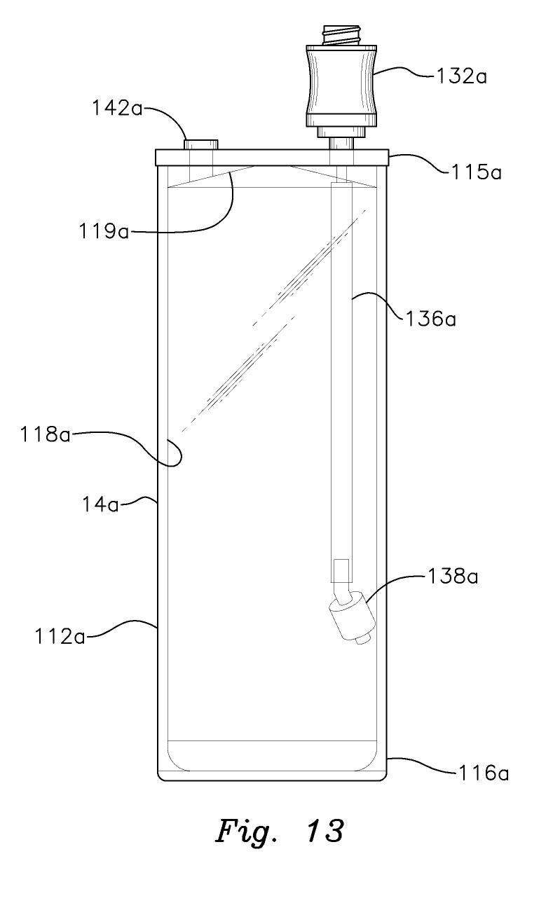

Aspiration ports 132 and 134 communicate with interior chamber 118. The interior end of plasma port 132 includes a fitting 161 that is communicatively engaged with an elongate aspiration pipe 136. The lower end of pipe 136 carries and is communicatively connected to a distal nozzle 138. Various types of nozzles suitable for connecting, either unitarily or separately, to the lower end of the aspiration pipe of the concentrating tube will be known to persons skilled in the art and may be employed within the scope of this invention. Although in the embodiments depicted herein the nozzle fits into the lower end of the aspiration pipe (See FIGS. 3 and 13), In certain alternative embodiments, the nozzle may simply be defined by the lower open end of the aspiration pipe itself. Aspiration pipe 136 is positioned within chamber 118 so that nozzle 138 is located above a depth within the chamber wherein a white buffy coat layer of platelets and white blood cells are formed during the separation process described below. As previously indicated, tube 112 is a modified version of the centrifuge tube currently available under the brand name Sequire™ manufactured and distributed by Emcyte Corporation. The details of that product, as well as the description contained in Patent No. '353 relating thereto are incorporated herein by reference. Such versions may alternatively be employed as the concentrating tube in the method of this invention. Nonetheless, tube 113 as shown in FIG. 3 and described herein, is particularly preferred for use as the concentrating tube. Another especially preferred concentrating tube is described below in FIGS. 13-17.

Preparation tube 12 and concentrating tube 112 are utilized to manufacture a highly concentrated or “pure” PRP product for medical applications in accordance with the method M set forth in FIG. 1. A step by step description of that method is further illustrated in FIGS. 4-11. In particular, the medical personnel performing the process initially assemble and arrange the required components to perform the method, including preparation tube 12, concentrating tube 112, a conventional centrifuge machine of the type normally employed to manufacture PRP, and assorted syringes that are suitable for loading and aspirating the centrifuge tubes according to the process of this invention. Again, various brands of standard and commercially available equipment used in this field may be employed. Nonetheless, the above-described centrifuge tubes manufactured and distributed by Emcyte Corporation are especially effective for use in the inventive process.

The system and method of this invention may be used to process various volumes of whole blood to produce a highly concentrated volume of PRP. Due to the size parameters and specifications of the centrifuge tubes, available syringes and standard centrifuge machines, a 50 ml whole blood sample is a convenient and practical volume to process in accordance with this invention. Referring to the diagram of FIG. 1 and the step by step illustrations of FIGS. 4-11 the above described equipment is utilized to process a 50 ml sample of whole blood from a patient in the following manner.

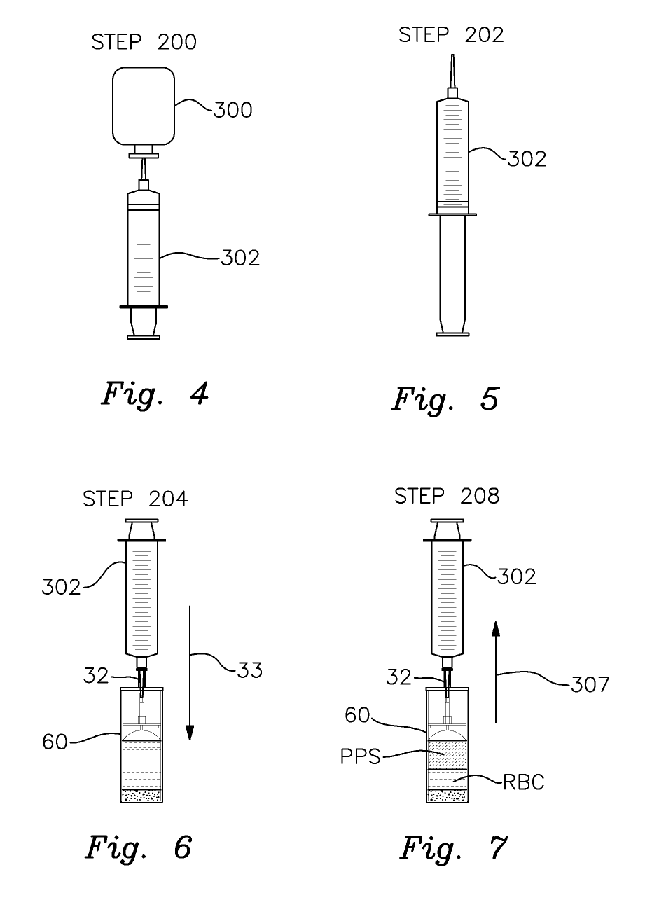

First, as shown in FIGS. 1 and 4, 10 ml of sodium citrate anticoagulant 300 is drawn into the chamber of a 60 ml syringe 302, step 200. A 50 ml whole blood sample from a patient to be treated is then drawn into syringe 302, step 202, and mixed with the sodium citrate in the syringe, step 203 (FIGS. 1 and 5). This fills syringe 302. The anticoagulated whole blood sample is then loaded into preparation tube 12, step 204, through the common port 32 as indicated by arrow 33. This drives the diaphragm 60 of tube 12 upwardly within the chamber of the tube as best shown in FIG. 6 such that the whole blood product 304 is contained within the interior chamber of tube 12 beneath the raised diaphragm 60. Syringe 302 is then disengaged from tube 12 and port 32 is capped or closed as indicated by engaged closure 38a in FIG. 2.

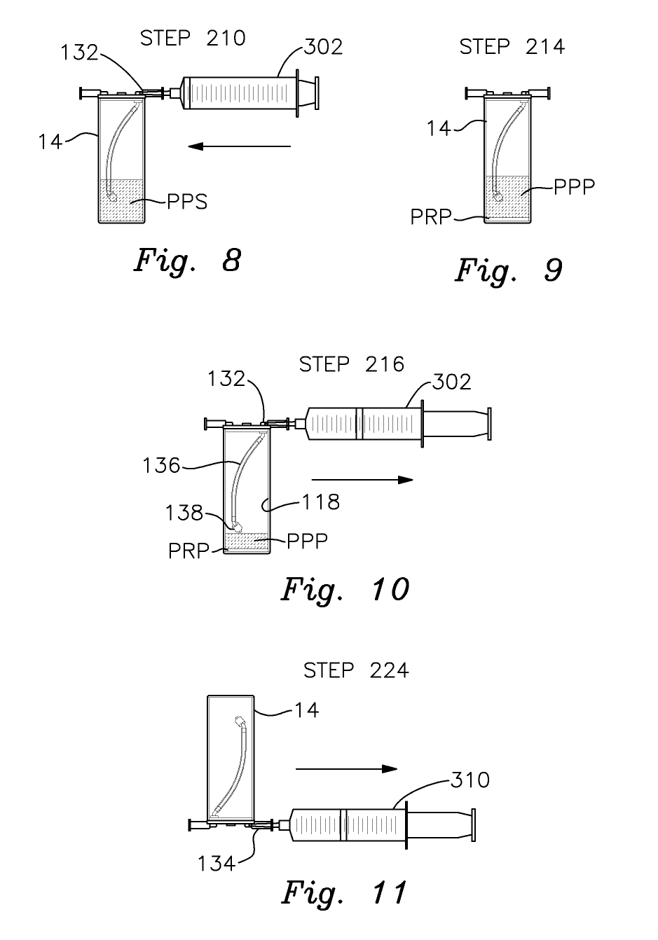

The capped tube 12 is loaded in a known manner into a standard centrifuge machine, which is suitable for centrifuging and separating blood products and similar applications. An Executive Series Centrifuge II™ machine or similar apparatus may be used for this purpose. The preparation tube is counterbalanced by placing a comparable volume of liquid in a second tube and installing that tube directly opposite tube 12 in the rotor of the centrifuge machine. The centrifuge lid is closed and the machine is set to a preferred speed of 3800 RPM and for an operating time of 1.5 minutes. These specifications may be varied within the scope of this invention as described below. The centrifuge machine is operated, step 206 in FIG. 1, at the selected speed and for the chosen time duration. Tube 12 is then removed from the centrifuge machine. Following this initial centrifuging process, the blood product in tube 12 has separated into a first upper level of fluid containing primarily a platelet plasma suspension (PPS) and a second lower fluid layer containing primarily red blood cells (RBC). A small amount of red blood cells will likely remain in the upper layer PPS. See FIGS. 1 and 8. The centrifuge machine should be set to operate in step 206 at a speed and for a time duration that separate most (more than 50%) of the red blood cells from the PPS and which allow at least 30% of the platelets in the blood sample to remain in the PPS layer.

As shown in FIGS. 1 and 8, syringe 302 is re-engaged with port 32 of tube 12. The engaged syringe is then operated to aspirate the entire platelet plasma suspension (PPS) layer into syringe 302, step 208 as indicated by arrow 307. Syringe 302 is then disengaged from tube 12 and the tube and remaining red blood cells (RBC) may be discarded.

Syringe 302 is next operatively attached to plasma port 132 of concentrating tube 14. See FIG. 9. Specifically, the cap 135, FIG. 3, is removed from port 132 and syringe 302, which contains the aspirated PPS, is attached to the plasma port. The syringe is then operated to transfer or load the plasma platelet suspension through plasma port 132, communicably connected aspiration pipe 136 and distal nozzle 138 into interior chamber 118 of concentrating tube 14, step 210 (FIGS. 1 and 9). Syringe 302 is then disengaged from port 132 and is recapped with a sterile dead-end cap.

Concentrating tube 14, which contains the platelet plasma suspension (PPS) is next fully capped by reattaching cap 135 to port 132, as indicated by engaged cap 135a (FIG. 3). Throughout the process to this point, cap 137 should remain engaged with PRP port 134, as indicated by attached cap 137a (FIG. 3). The fully capped tube 14 is placed in the rotor of the standard centrifuge machine, and counterbalanced in the manner previously described. The centrifuge machine lid is closed and the machine is set to a preferred speed of 3800 RPM and operating time of 5 minutes. The centrifuge machine is then operated, step 212, such that the PPS is separated, step 214, into two concentrated stage fluid layers in tube 14, namely an upper fluid layer comprising primarily a platelet poor plasma (PPP) and a lower layer comprising primarily a platelet concentrated buffy coat (PRP). More particularly, the centrifuge is set to operate at a speed and duration that cause additional red blood cells to separate from the plasma and collect in the PPP layer. The speed and duration of the centrifuge machine also cause at least, and preferably more than, 50% of the platelets from the PPS to be retained in the PRP layer.

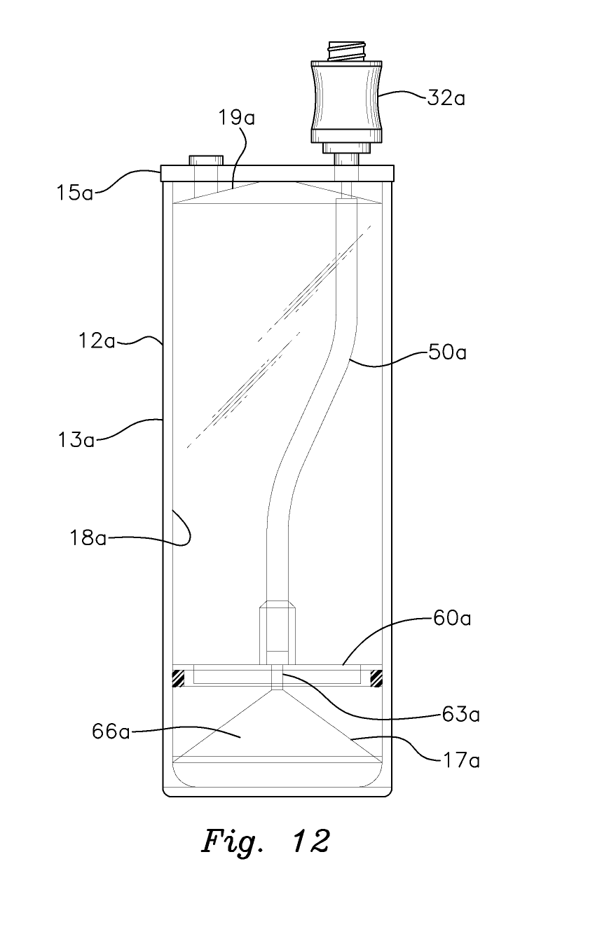

After the concentrating tube 14 has undergone the second centrifuging stage, it is removed from the centrifuge machine. Most, if not all of the PPP layer is located above the distal nozzle 138 of aspiration pipe 136. By the same token, most if not all of the PRP layer is located below nozzle 138, step 214 (FIGS. 1 and 11). Cap 135 is then removed from plasma port 132. Syringe 302 is operatively engaged with open plasma port 132 and the syringe is operated to aspirate the upper PPP layer from chamber 118 of tube 14, step 216 (FIGS. 1 and 12). Due to the positioning of nozzle 138 in chamber 118, most, if not all of the upper PPP layer is aspirated and most if not all of the lower PRP layer (i.e. 7 ml) remains within tube 14. Syringe 302 is disconnected from port 132 and the plasma port is recapped with closure 135x (FIG. 3). The aspirated PPP fluid may be discarded.

The PRP remaining in the bottom of tube 14 is in the form of a white, highly platelet concentrated plasma in the form of a whitish buffy coat containing trace amounts of red blood cells. Plasma may be added to PRP level via plasma port 132 to dilute the PRP and attain more volume if necessary. After the addition of additional plasma, port 132 is re-capped and the platelet buffy coat is re-suspended into the added plasma by gently swirling or agitating the device, step 220. As shown in FIGS. 1 and 13, concentrating tube 14 is then inverted, step 222. The closure 137 is removed from PRP port 134 and a 12 ml syringe 310 is operatively attached to port 134. Syringe 310 is then operated to aspirate the remaining PRP from tube 14, step 224. Syringe 310 is disengaged from port 134 and a sterile cap is attached to the syringe.

Syringe 310 contains the highly platelet concentrated PRP which will contain only a very small, trace concentration of red blood cells (i.e. 1% or less). This is a significant reduction of red blood cells from the amount typically present in PRP manufactured in accordance with conventional, prior art principals. In such cases, red blood cell concentrations of 15% or more are commonplace. The significant reduction in red blood cell concentration and the proportionately increased concentration of medically effective platelets provides for a significantly improved and more medically effective PRP product. The highly concentrated and effectively “pure” PRP produced by the method of this invention is achieved only by using the novel two stage centrifuging process and system set forth by this invention. It is particularly important that the centrifuging parameters of speed and time by followed so that separation of the constituent blood components occurs in a sequence that effectively separates a greater percentage of red blood cells from the PRP while retaining a greater proportion of platelets than has heretofore been accomplished. As a result, a high quality and therapeutically superior product is obtained which can be used in a wide variety of surgical, wound care, dental, bone regeneration and other medical applications. The manufacturing process is also commercially efficient and allows high quality and highly concentrated PRP to be produced using commercially available equipment and without the need for technically complicated and/or unavailable equipment and procedures.

In alternative embodiments, the various centrifuge tubes disclosed in Patent No. '353 may be used to separate the platelet plasma suspension into PPP and PRP layers. Those layers can then be sequentially aspirated without inverting the tube, either through respective aspiration pipes or using a single sliding pipe as disclosed by that reference. Typically, in these versions the concentrating tube is centrifuged twice as disclosed in Patent No. '353. By the same token, the Progenikine™ tube disclosed in Ser. No. 14/741,920 may be employed as the preparation tube and utilized as disclosed in that reference to prepare the platelet plasma suspension that is introduced into the concentration tube. The process of the invention nonetheless achieves particularly preferred results (i.e. improved platelet concentrations and reduced red blood cell concentrations) by employing the centrifuge operating parameters (speed and duration) and the concentrating tube specifications described herein by operating the centrifuge for only 1% minutes of approximately 3800 rpm during centrifugation of the preparation tube. Two discrete fluid layers (platelet plasma suspension (PPS) and red blood cells (RBC)) are produced rather than the three discrete layers created by centrifuging blood product for 10 minutes, as has been performed previously. The PPS is capable of being further separated and concentrated within the concentrating tube, as described herein, so that increased platelet and greatly reduced red blood cell concentrations are achieved.

As shown in FIG. 12, a slightly modified preparation tube 12a includes a cylindrical receptacle 13a enclosing an interior chamber 18a. A permanently capped upper end 15a includes a conically tapered inner surface 19a. A common inlet/outlet port comprising a conventional self-sealing valve port 32a, which will be known to persons skilled in the art of centrifuge tubes, communicates with interior chamber 18a through upper end 15a. An aspiration pipe 50a is connected to a sealing diaphragm or piston 60a and communicates with a lower interior portion 66a of the chamber through a tubular channel 63a. In this version, diaphragm 60a includes a generally conically shaped bottom surface 17a. Otherwise, the construction of preparation tube 12a is identical or closely analogous to that of previously described preparation tube 12. The previously described outlet cap and connecting strap are omitted in FIG. 12 but may be engaged with the preparation tube particularly during centrifugation.

An alternative concentrating tube 14a is depicted in FIG. 13. Once again, this tube employs a cylindrical receptacle 112a having permanently capped upper and lower ends 115a and 116a, respectively. The receptacle encloses an interior chamber 118a. Upper end 115a includes a conically tapered interior surface 119a.

In this version, concentrating tube 14a includes a single, common inlet and aspiration or outlet port 132a, which again comprises a self-sealing valve port, which is standard in the centrifuge industry. Port 132a and a pressure neutralizing vent 142a are formed through the upper end 115a of receptacle 112a in communication with interior chamber 118a. More particularly, a flexible aspiration pipe 136a is communicably connected to the inner end of port 132a and extends downwardly through chamber 118a such that a nozzle 138a carried at the lower end of pipe 136a is positioned within the platelet poor plasma layer of the aspirated platelet plasma suspension following centrifugation of the concentrating tube. More particularly, nozzle 138 is angled such that it generally points toward and is proximate the sidewall of receptacle 112a. This positioning of the nozzle is particularly effective for performing the process of this invention as described more fully below.

The process of this invention may be performed using any of the preparation tubes described herein in combination with the concentrating tube shown in FIG. 13. For example, in a preferred embodiment, 50 ml (or some other volume) of whole blood is drawn from a patient and filled into a 60 ml. syringe, which also contains 10 ml of sodium citrate anticoagulant. The anticoagulated whole blood is then loaded into the preparation tube and centrifuged (preferably for 1.5 minutes at 3800 rpm) so that the blood product is separated into an upper platelet plasma suspension (PPS) layer and a lower red blood cell (RBC) layer. The platelet plasma suspension is then aspirated from the preparation tube as previously described. PPS is aspirated in this manner until RBC is drawn into the aspirating pipe 136, 136a. It is normal to aspirate small amounts of RBC into the syringe during this step of the process. The aspirated PPS is then loaded into concentrating tube 14a through common inlet/outlet port 132a, communicably attached pipe 136a and nozzle 138a. Tube 114a is then placed in the centrifuge machine and counterbalanced. The centrifuge machine is operated for 5 minutes at 3800 rpm to separate the PPS within tube 114a into an upper platelet poor plasma (PPP) layer, containing at most and preferably less than 50% of the platelets from the PPS, and a lower, platelet rich plasma (PRP) layer, which contains at least and preferably more than 50% of the platelets from the PPS in a manner analogous to that previously depicted in FIG. 9.

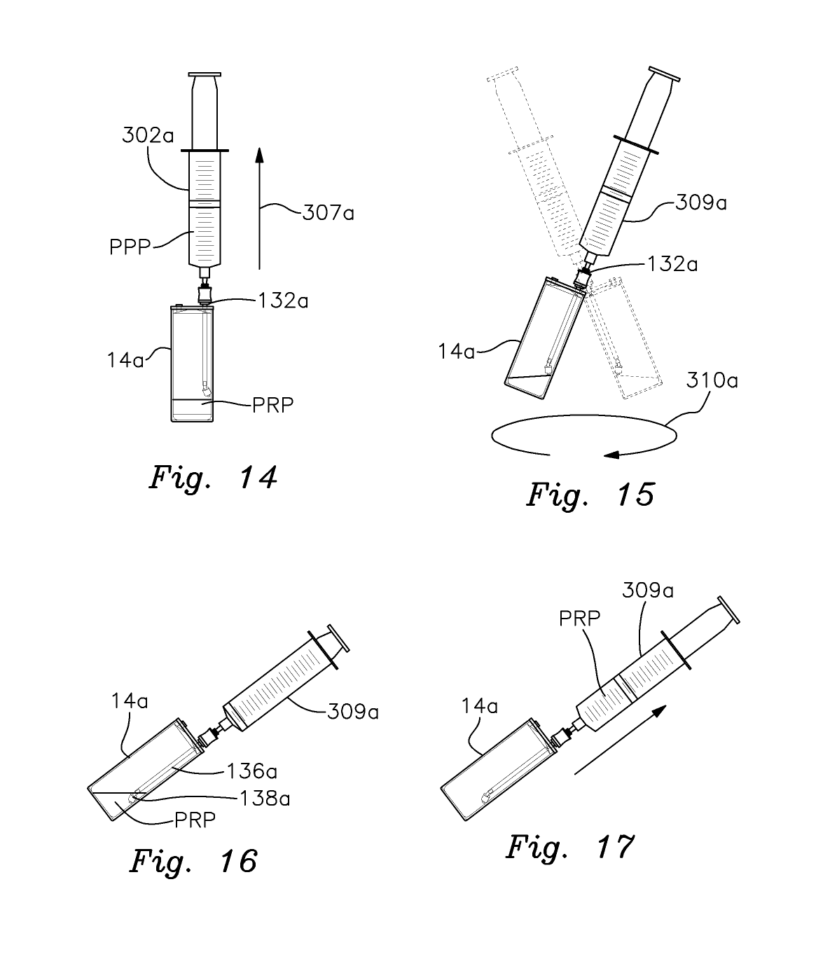

As shown in FIG. 14, a syringe 302a is engaged with port 132a of tube 14a and the syringe is operated to draw PPP out of tube 14a as indicated by arrow 307a. Once again, the nozzle at the lower end of pipe 136a is held at a height within tube 14a that is within and preferably close to the bottom of the PPP layer within the tube. This causes most of the PPP layer to be aspirated and removed through nozzle 138a, attached aspiration pipe 136a and port 132a. In this example, approximately 7 ml of PRP remains at the bottom of tube 14a.

Next, an empty 12 ml syringe 309a is attached to the port 132a as shown in FIG. 15. The concentrating tube 114a is swirled, as indicated by arrow 310a, or otherwise agitated to re-suspend the platelet buffy coat PRP into the plasma remaining within the tube. As previously indicated, additional plasma may be added to tube 114a (through common inlet and outlet port 132a) if desired.

After the platelets have been re-suspended within the plasma, the concentrating tube 114a is tilted as shown in FIG. 1, step 223, and in FIG. 16. This pools the remaining PRP into a lower corner of the tube and causes the distal nozzle 138a carried at the lower end of pipe 136 to immerse within the pooled PRP. Syringe 309a is then drawn to aspirate the PRP that remains within tube 114a. The flexibility of pipe 136a and the proximity of angled nozzle 138a to the sidewall of the concentrating tube (see FIG. 13) facilitate immersion of the nozzle into the concentrated PRP. The aspiration draws most, if not all, of the concentrated PRP out of the concentrating tube 114a as indicated in FIG. 17. A highly concentrated PRP is drawn into syringe 309a. This PRP may then be used effectively in various medical procedures. Typically, a concentration of 1% or less of red blood cells remains in the purified and highly concentrated PRP product.

The use of the anticoagulant sodium citrate in PRP production also constitutes a novel and significantly improved feature of this invention. To date, the anticoagulant

ACDA has been universally used in the production of PRP in order to prevent blood clots, which can interfere with effective separation of the blood constituent components during the production process. ACDA is fairly acidic and features a pH of approximately 6.8-7.2. This causes PRP that has been treated with ACDA to have very painful side effects for the patient. Nonetheless, ACDA is virtually always used as an anticoagulant because it includes dextrose, a food source which allows the blood product to be stored for up to 30 days. I have determined that such storage is unnecessary in perfusion and PRP applications inasmuch as the produced PRP is almost always used at the point of patient care. It is usually unnecessary to store the PRP product for any length of time at all. Accordingly, the dextrose contained in ACDA is not needed in such applications. Sodium citrate, on the other hand, lacks dextrose and features a normal blood pH of 7.35-7.45. As a result, it does not exhibit the painful patient side effects when used as an anticoagulant for PRP. Sodium citrate is therefore less painful and a much more effective anticoagulant to use when performing medical procedures involving PRP therapy.

From the foregoing it may be seen that this invention provides for a method and system for more effectively concentrating blood platelets for use in medical applications. While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

What is claimed is:

1. A method of producing a platelet rich plasma (PRP) having a concentrated level of platelets and a reduced level of red blood cells, said method comprising:
providing a preparation centrifuge tube that includes an elongate receptacle having an interior chamber for receiving a blood product therein, which receptacle has closed upper and lower ends and a side wall extending between said upper and lower ends;
providing a liquid impermeable sealing diaphragm within said chamber such that said diaphragm is longitudinally slidable through said chamber and maintains sealing interengagement with an interior surface of said side wall of said receptacle, said diaphragm separating said chamber into said upper and lower chamber regions, said preparation centrifuge tube further including a common inlet and outlet port formed through said receptacle and communicably connected to one of said upper and lower chamber regions, said preparation centrifuge tube further including a vent formed through said receptacle and communicably connected to the other of said upper and lower chamber regions for neutralizing air pressure within said receptacle;
providing a concentrating tube that includes closed upper and lower end portions and a sidewall extending between said upper and lower end portions, said concentrating tube further having first and second aspiration ports formed through said upper end portion and communicably connected to an interior chamber of said concentrating tube, at least said first aspiration outlet having an aspiration pipe joined communicably thereto and extending to a distal nozzle at a predetermined depth within said chamber;
introducing a blood product into one of said upper and lower chamber regions of said preparation tube through said common inlet and outlet port to drive said ceiling diaphragm longitudinally through said chamber;
centrifuging said preparation tube at a speed and for a duration that separates the blood product into discrete fluid layers including an upper platelet plasma suspension layer and a lower red blood cell layer;
aspirating said platelet plasma suspension layer from said preparation tube and introducing said platelet plasma suspension into the chamber of said concentrating tube;
centrifuging the concentration tube at a speed and for a duration that separates the platelet plasma suspension into an upper platelet poor plasma layer containing a minority of the platelets from the platelet plasma suspension and a platelet rich plasma layer that includes a majority of the platelets from the platelet plasma suspension;
aspirating the platelet poor plasma layer from said concentrating tube through said first aspirating port; and
aspirating platelet rich plasma layer from the concentrating tube through said second aspirating port.

2. The method of claim 1 further including the step of mixing the blood product with an anticoagulant comprising sodium citrate.

3. The method of claim 2 in which said anticoagulant is mixed with said blood product in a ratio of 1:5 parts by volume.

4. The method of claim 1 in which said preparation tube is centrifuged at a speed and for a duration that cause at least 30% of platelets in the blood sample to remain in the platelet plasma suspension and remove more than 50% of the end blood cells from the platelet plasma suspension.

5. The method of claim 1 in which said aspirated platelet plasma suspension is introduced into said concentrating tube through said first aspiration port.

6. The method of claim 1 in which said concentrating tube is centrifuged for a time and duration such that the lower, platelet rich plasma layer is disposed mostly, if not entirely below, the nozzle of said aspiration pipe and the upper platelet poor plasma layer disposed mostly, if not entirely above, the nozzle of said aspiration pipe.

7. The method of claim 1 further including the step of adding plasma to the concentrating tube following aspiration of the platelet poor plasma layer from the concentrating tube to dilute and add to the volume of the platelet rich plasma layer and agitating the concentrating tube to re-suspend the platelets of the platelet rich plasma into the added plasma prior to aspirating the platelet rich plasma layer from said concentrating tube.

8. The method of claim 1 further including the step of inverting the concentrating tube after the platelet poor plasma layer has been aspirated from the concentrating tube and aspirating the platelet rich plasma layer through the second aspiration port.

9. The method of claim 1 further including the step of agitating the concentration tube to re-suspend platelets in the platelet rich plasma layer before the platelet rich plasma layer is aspirated from the concentrating tube.

10. A method of producing a platelet rich plasma (PRP) having a concentrated platelet level and a reduced level of red blood cells, said method comprising:
providing a preparation centrifuge tube that includes an elongate receptacle having an interior chamber for receiving a blood product therein, which receptacle has closed upper and lower ends and a side wall extending between said upper and lower ends;
providing a liquid impermeable sealing diaphragm within said chamber such that said diaphragm is longitudinally slidable through said chamber and maintains sealing interengagement with an interior surface of said side wall of said receptacle, said diaphragm separating said chamber into upper and lower regions;
providing said preparation centrifuge tube further with a common inlet and outlet port formed through said receptacle and communicably connected to one of said upper end lower chamber regions and a vent formed through said receptacle and communicably connected to the other of said upper and lower chamber regions for neutralizing air pressure within said receptacle;
providing said concentrating tube with a receptacle that has an interior chamber including closed upper and lower ends and a sidewall extending between the upper and lower ends;
further providing said concentrating tube with plasma and PRP aspiration ports proximate the upper end of the receptacle and an aspiration pipe communicably connected to said plasma aspiration port and extending through said chamber of said concentrating tube to a distal nozzle at a predetermined depth within said chamber;
introducing a blood product into one of said upper and lower regions of said preparation tube through said common inlet and outlet port to drive said diaphragm longitudinally through said chamber;
centrifuging the preparation tube at a speed and for a duration that separates the blood product into discrete fluid layers including an upper platelet plasma suspension and a lower red blood cell layer;
aspirating the upper platelet plasma suspension layer from said chamber of the receptacle of the preparation tube;
introducing the aspirated upper fluid layer into the chamber of the concentrating tube through the plasma port of the concentrating tube;
centrifuging the concentrating tube at a speed and for a duration that separates the platelet plasma suspension into discrete upper and lower layers that respectively include a platelet poor plasma layer retaining less than 50% of the platelets from the platelet plasma suspension and a platelet rich plasma layer retaining more than 50% of the platelets from the platelet plasma suspension;
aspirating the platelet poor plasma from said chamber of said concentrating tube through said aspiration pipe and said plasma aspiration port;
inverting the receptacle of said concentration tube; and
aspirating the platelet rich plasma from said chamber of said concentrating tube through said PRP aspiration port.

11. The method of claim 9 in which the preparation tube is centrifuged at a speed and for a duration such that the platelet plasma suspension layer retains at least 30% of the platelets from the blood product.

12. The method of claim 10 further including the step of mixing the blood product with an anticoagulant comprising sodium citrate.

13. The method of claim 12 in which said anticoagulant is mixed with said blood product in a ratio of 1:5 parts by volume.

14. The method of claim 10 in which said concentrating tube is centrifuged for a time and duration such that upper platelet poor plasma layer is disposed mostly, if not entirely above the distal nozzle of said aspiration pipe and the lower, platelet rich plasma layer is disposed mostly, if not entirely below the lower end of said aspiration pipe.

15. The method of claim 10 further including the step of adding plasma to the concentrating tube following aspiration of the platelet poor plasma layer from the concentrating tube to dilute and add to the volume of the platelet rich plasma layer and agitating the concentrating tube to re-suspend the platelets in the added plasma.

16. The method of claim 10 further including the step of agitating the concentration tube to re-suspend platelets in the platelet rich plasma layer before the platelet rich plasma is aspirated from the concentrating tube.

17. The method of claim 1 in which the platelet rich plasma aspirated form the concentrating tube includes a red blood cell concentration of not greater than 1%.

18. The method of claim 10 in which the platelet rich plasma aspirated form the concentrating tube includes a red blood cell concentration of not greater than 1%.

19. A method of producing a platelet rich plasma (PRP) having a concentrated platelet level and a reduced level of red blood cells, said method comprising:
providing a preparation centrifuge tube that includes an elongate receptacle having an interior chamber for receiving a blood product therein, which receptacle has closed upper and lower end portions and a side wall extending between said upper and lower end portions;
providing a liquid impermeable sealing diaphragm within said chamber such that said diaphragm is longitudinally slidable through said chamber and maintains sealing interengagement with an interior surface of said side wall of said receptacle, said diaphragm separating said chamber into upper and lower regions;
providing said preparation centrifuge tube further with a common inlet and outlet port formed through said receptacle and communicably connected to one of said upper end lower chamber regions and a vent formed through said receptacle and communicably connected to the other of said upper and lower chamber regions for neutralizing air pressure within said receptacle;
providing said concentrating tube with a receptacle that has an interior chamber including closed upper and lower ends and a sidewall extending between the upper and lower ends;
further providing said concentrating tube with a common inlet and outlet aspiration port proximate the upper end of the receptacle and an aspiration pipe communicably connected to said common inlet and outlet port of said concentrating tube and extending through said chamber of said concentrating tube to a distal nozzle at a predetermined depth within said chamber;
introducing a blood product into one of said upper and lower regions of said preparation tube through said common inlet and outlet port of said preparation tube to drive said diaphragm longitudinally through said chamber;
centrifuging the preparation tube at a speed and for a duration that separates the blood product into discrete fluid layers including an upper platelet plasma suspension and a lower red blood cell layer;
aspirating the upper platelet plasma suspension layer from said chamber of the receptacle of the preparation tube;
introducing the aspirated upper fluid layer into the chamber of the concentrating tube through the common inlet and outlet port of the concentrating tube;
centrifuging the concentrating tube at a speed and for a duration that separates the platelet plasma suspension into discrete upper and lower layers that respectively include a platelet poor plasma layer retaining less than 50% of the platelets from the platelet plasma suspension and a platelet rich plasma layer retaining more than 50% of the platelets from the platelet plasma suspension;
aspirating the platelet poor plasma from said chamber of said concentrating tube through said aspiration pipe and said common inlet and outlet port of said concentrating tube;
tilting the receptacle of said concentrating tube such that the distal nozzle carried by said aspiration pipe is immersed in the platelet rich plasma remaining in the concentrating tube; and
aspirating the platelet rich plasma from said chamber of said concentrating tube through the nozzle, aspiration pipe and common inlet and outlet of the concentrating tube.

20. The method of claim 19 further including the step of agitating the concentration tube to re-suspend platelets in the platelet rich plasma layer before the platelet rich plasma is aspirated from the concentrating tube.
